(19) United States
(12) Patent Application Publication
(10) Pub. No.: US 2016/0115113 A1
(43) Pub. Date: Apr. 28, 2016

(54) Aerobic Oxidative Esterification of Sugar-Derived 1,4-Disubstituted Benzene for Direct Synthesis of Dimethylterephthalate

(71) Applicant: ExxonMobil Chemical Patents Inc., Baytown, TX (US)

(72) Inventors: Mika Shiramizu, Houston, TX (US); James R. Lattner, La Porte, TX (US); Kapil Kandel, Humble, TX (US)

(21) Appl. No.: 14/877,545

(22) Filed: Oct. 7, 2015

Related U.S. Application Data
(60) Provisional application No. 62/069,508, filed on Oct. 28, 2014.

(51) Int. Cl. C07C 67/03; C07C 37/52

(52) U.S. Cl. CPC C07C 67/03; C07C 37/52

(57) ABSTRACT

This invention relates to a dimethylterephthalate production process comprising reacting substituted furan with ethylene under cycloaddition reaction conditions and in the presence of a cycloaddition catalyst to produce a bicyclic ether, dehydrating the bicyclic ether to produce a substituted phenyl, dissolving said substituted phenyl in methanol, and oxidizing and esterifying the substituted phenyl in the presence of an oxidative esterification catalyst to form dimethylterephthalate. Importantly, the process does not include oxidizing the substituted phenyl to form terephthalic acid.

### PRIORITY

This invention claims priority to and the benefit of U.S. Ser. No. 62/069,508, filed Oct. 28, 2014.

### FIELD OF THE INVENTION

The present invention relates to a process for the production of dimethylterephthalate. The invention relates more particularly to overall biobased pathways for making dimethylterephthalate from carbohydrates such as hexoses (e.g., glucose or fructose).

### BACKGROUND OF THE INVENTION

Terephthalic acid and its methyl esters are useful in the production of various polymers such as poly(ethylene terephthalate), poly(propylene terephthalate), and poly(butene terephthalate). Terephthalate polymers, such as poly(ethylene terephthalate) (PET), have many uses such as, for example, for making synthetic fibers and food-grade containers (e.g., beverage bottles). Major sources of terephthalic acid include oxidation of para-xylene streams that result from the refining of crude oil. However, the oxidation requires the use of a highly corrosive homogeneous catalyst and co-produces 4-carboxybenzaldehyde which cannot be readily removed by distillation or crystallization thereby requiring an additional reaction step before polymer grade terephthalic acid can be produced.

In addition, growing concerns about the high costs of production of hydrocarbon fuel components and petrochemicals, such as para-xylene, have attracted attention to alternate sources such as renewable feedstocks. Renewable biomass resources are useful in the synthesis of substitutes for petroleum-derived products and there is an ongoing need for processes to synthesize, from bio-based feedstocks, additional compounds that are traditionally products of the petroleum and/or petrochemical industries. However, the difficulty in converting natural 6-carbon carbohydrate building blocks, such as glucose or fructose, to desirable end products has hindered progress in some important areas. Recent studies have shown the feasibility of converting hexose carbohydrates to 2,5-dimethylfuran (DMF). For example, Leshkov, Y. R. et al. report the production of 5-hydroxymethylfurfural (HMF) in high yields by the acid catalyzed dehydration of fructose, followed by the selective hydrogenation of HMF to DMF using a copper-based catalyst (NATURE, June 2007, (447): pp. 982-5). Also, Zhao, H. et al. describe the synthesis of HMF, starting with glucose, in the presence of a metal halide (e.g., chromium (II) chloride) in 1-alkyl-3-methylimidazolium chloride (SCIENCE, June 2007, (3 16): pp. 1597-1600).

In our co-pending U.S. Patent Application Ser. No. 61/898,521 filed Nov. 1, 2013, we have described a process for the conversion of substituted furan (SF) compounds, particularly 5-hydroxymethylfurfural (HMF) or 2,5-bis hydroxymethylfuran (BHMF), to terephthalic acid using a Diels-Alder cycloaddition reaction with ethylene. The cycloaddition reaction produces a bicyclic ether which is then dehydrated to 1,4-dihydroxymethylbenzene (DHMB) or 4-(hydroxymethyl)benzaldehyde (HMBA), which in turn can be oxidized to terephthalic acid. The similar reaction can be envisioned for the conversion of 2,5-diformylfuran, the partially oxidized form of HMF, to terephthalaldehyde (TPal). Advantageously, the SF starting material for the process may be synthesized from carbohydrates, thereby providing a production route to terephthalic acid that relies at least partly on renewable feedstocks.

U.S. Pat. No. 7,385,081 describes the synthesis of terephthalic acid from carbohydrate derivatives. HMF is first oxidized to furan dicarboxylic acid (FDCA), which can then be esterified to 2,5-furan dicarboxylate. Ethylene is reacted with the FDCA or the furan dicarboxylate to form a Diels-Alder cycloadduct, which is then dehydrated to terephthalic acid or the terephthalic ester. This is referred to as Route I.

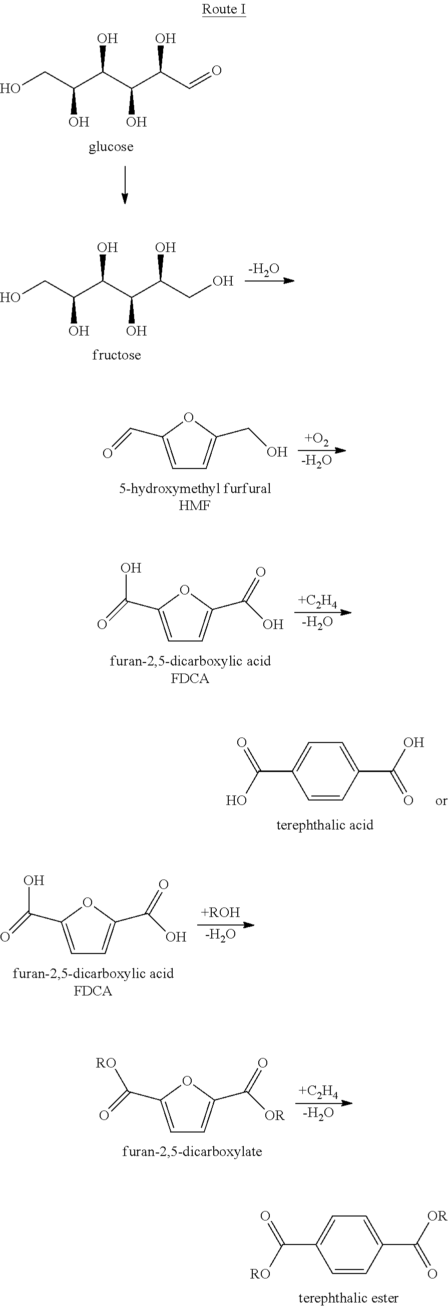

WO2010/151346 discloses the production of para-xylene by reacting DMF with ethylene under cycloaddition reaction conditions and in the presence of a catalyst. The p-xylene produced from this route can then be oxidized to terephthalic acid. This is referred to as Route II.

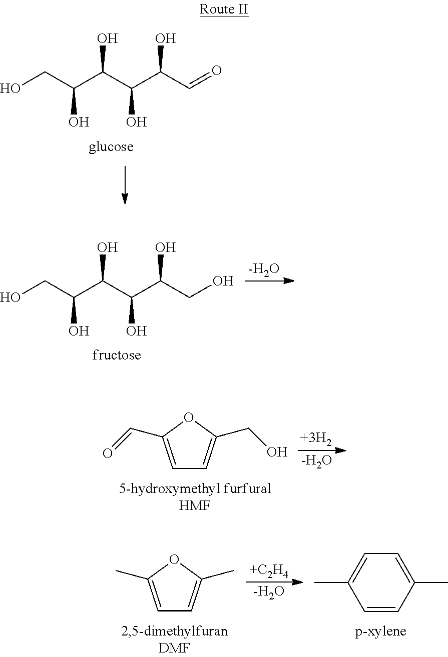

American Chemical Society Catalysis 2012, 2, pp. 935-939 discloses conversion of HMF to dimethylfuran which is then converted by ethylene cycloaddition to para-xylene.

Jagadeesh, et al. (“Selective Oxidation of Alcohols to Esters Using Heterogeneous Co₃O₄—N@C Catalysts under Mild Conditions”, Journal of the American Chemistry Society, 2013, 135, pp. 10776-10782) discloses oxidation of DHMB to DMTP in 89% yield using Co catalyst supported on carbon material, methanol as solvent/reactant, in combination with 0.4 eq. of K₂CO₃ base. The reaction was conducted under 1 bar O₂ at 60° C. for 24 hr.

Li et al. (“From Alkyl Aromatics to Aromatic Esters: Efficient and Selective C-H Activation Promoted by a Bimetallic Heterogeneous Catalyst”, Chem Sus Chem, 2012, 5, pp. 1892-1896) discloses the oxidation of p-xylene to methyl 4-methylbenzoate in 80% yield (120° C., 1.0 MPa O₂, 72 h) and the oxidation of methyl 4-methylbenzoate to DMTP in 65% yield (140° C., 1.0 MPa O₂, 48 h) using Au-Pd bimetallic catalyst supported on metal organic framework MIL-101, and methanol as solvent/reactant.

Menegazzo et al. (Journal of Catalysis 2014, 309, pp. 241-247), among others, discloses aerobic oxidative esterification of furfural, HMF, 2,5-diformylfuran using mostly supported gold catalysts.

Additional references of interest include: WO2012/125218, WO2013/040514, and WO2013/048248. Methods for preparing terephthalic acid and terephthalic esters from certain biomass-derived starting materials are mentioned in WO2010/148081 and WO2010/151346.

It would be advantageous if dimethyl terephthalate could be synthesized from renewable sources of carbon and in the absence of terephthalic acid.

### SUMMARY OF THE INVENTION

According to the present invention, it has now been found that substituted phenyl intermediates, which can be derived from sugars, can be oxidized and esterified to dimethylterephthalate (DMTP) by dissolving the intermediates in methanol and contacting the solution with O₂ or air under mild catalytic conditions.

In a first embodiment, this invention relates to a dimethylterephthalate production process comprising reacting substituted furan with ethylene under cycloaddition reaction conditions and in the presence of a cycloaddition catalyst to produce a bicyclic ether, wherein the substituted furan is represented by the formula:

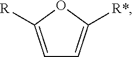

wherein R and R* are separately selected from CH₃, HC═O and —CH₂OH, and the bicyclic ether is represented by the formula:

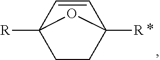

then dehydrating the bicyclic ether to produce a substituted phenyl of the formula:

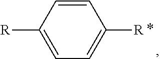

then dissolving said substituted phenyl in methanol, and oxidizing and esterifying the substituted phenyl in the presence of an oxidative esterification catalyst to form dimethylterephthalate.

Advantageously, the process of this embodiment can be conducted when both R and R* are HC═O, or when both R and R* are —CH₂OH, or when one of R and R* is HC═O and the other is —CH₂OH, or when both R and R* are CH₃, or when one of R and R* is CH₃ and the other is —CH₂OH, or when one of R and R* is CH₃ and the other is HC═O.

In a preferred embodiment, the oxidative esterification is conducted at a temperature from −50° C. to 250° C.

In one embodiment of the invention, the oxidative esterification catalyst is a heterogeneous catalyst bearing metal including but not limited to cobalt, gold, palladium, platinum, and combinations thereof.

In another embodiment, the oxidative esterification catalyst is supported on materials including but not limited to carbon, alumina, titania, silica, and metal-organic framework.

Preferably, the oxidative esterification is conducted at a substantially neutral pH or in the presence of a recoverable solid base catalyst.

In another embodiment, the substituted furan is derived from conversion of glucose or fructose, and preferably the process does not include oxidizing the substituted phenyl to form terephthalic acid.

In another embodiment, the invention is directed to a carbohydrate based process for producing dimethylterephthalate comprising (a) converting a hexose to 5-hydroxymethylfurfural, (b) reacting the 5-hydroxymethylfurfural with ethylene under cycloaddition reaction conditions and in the presence of a catalyst to produce a bicyclic ether, then dehydrating the bicyclic ether to produce a compound represented by the formula (I):

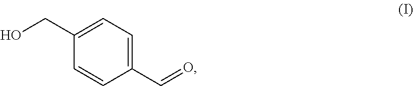

and (c) dissolving the compound represented by the formula (I) in methanol and contacting the solution with oxygen in the presence of an oxidation/esterification catalyst to produce dimethylterephthalate, wherein the compound of formula (I) is not converted to terephthalic acid prior to step (c).

In another embodiment, the invention is directed to a carbohydrate based process for producing dimethylterephthalate comprising (a) converting a hexose to 5-hydroxymethylfurfural, (b) hydrogenating 5-hydroxymethylfurfural to form 2,5-bis hydroxymethylfuran, (c) reacting the 2,5-bis hydroxymethylfuran with ethylene under cycloaddition reaction conditions and in the presence of a catalyst to produce a bicyclic ether, then dehydrating the bicyclic ether to produce a compound represented by the following formula (II):

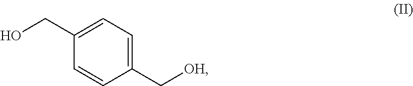

and (d) dissolving the compound represented by the formula (II) in methanol and contacting the solution with oxygen in the presence of an oxidation/esterification catalyst to produce dimethylterephthalate, wherein the compound of formula (II) is not converted to terephthalic acid prior to step (d).

In another embodiment, the invention is directed to a carbohydrate based process for producing dimethylterephthalate comprising, (a) converting a hexose to 5-hydroxymethylfurfural, (b) partially oxidizing the 5-hydroxymethylfurfural to form diformyl furan, (c) reacting the diformyl furan with ethylene under cycloaddition reaction conditions and in the presence of a catalyst to produce a bicyclic ether, then dehydrating the bicyclic ether to produce a compound represented by the following formula (III):

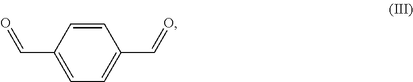

and (d) dissolving the compound represented by the formula (III) in methanol and contacting the solution with oxygen in the presence of an oxidation/esterification catalyst to produce dimethylterephthalate, wherein the compound of formula (III) is not converted to terephthalic acid prior to step (d).

In another embodiment, the invention is directed to a carbohydrate based process for producing dimethylterephthalate comprising, (a) converting a hexose to 5-hydroxymethylfurfural, (b) reducing the 5-hydroxymethylfurfural to form dimethyl furan, (c) reacting the dimethyl furan with ethylene under cycloaddition reaction conditions and in the presence of a catalyst to produce a bicyclic ether, then dehydrating the bicyclic ether to produce a compound represented by the following formula (IV):

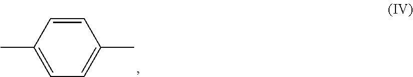

and (d) dissolving the compound represented by the formula (IV) in methanol and contacting the solution with oxygen in the presence of an oxidation/esterification catalyst to produce dimethylterephthalate, wherein the compound of formula (IV) is not converted to terephthalic acid prior to step (d).

### BRIEF DESCRIPTION OF THE DRAWINGS

FIG. 1 shows a preferred embodiment of the invention.

### DETAILED DESCRIPTION OF THE INVENTION

According to the present invention, it has now been found that p-xylene, DHMB, HMBA and TPal intermediates can be oxidized and esterified to dimethylterephthalate (DMTP) by dissolving the intermediates in methanol and contacting the solution with O₂ or air under mild catalytic conditions. The resulting crude DMTP is easily purified by distillation for use in the production of polyesters. The oxidative esterification of the p-xylene, DHMB, HMBA, or TPal starting materials has an advantage over the conventional p-xylene oxidation route to terephthalic acid, in that (i) the oxidizing conditions are milder and less corrosive, and (ii) the direct synthesis of the di-ester (as opposed to the di-acid) allows easier removal or impurities in the crude DMTP by distillation. That is, the process does not include oxidizing the substituted phenyl to form terephthalic acid.

In the conventional production of terephthalic acid, such as by oxidation of p-xylene, an additional reaction step (either esterification or hydrogenation) is required to purify the product to polymer-grade. The present invention eliminates this second reaction step because the dimethyl ester is synthesized directly from a 1,4-disubstitutedphenyl. The present invention is additionally advantageous because it may be conducted either under substantially neutral pH, or in the presence of a recoverable solid base catalyst. The catalyst and the process as a whole have enhanced stability and durability, due to the relatively mild conditions under which the process can be conducted. Additionally, due to the milder oxidation conditions, there is reduced loss of solvent. Advantageously, the source of carbon for the process is renewable, being hexose/sugar-derived.

As used herein, the new notation for the Periodic Table Groups is used as described in Chemical and Engineering News, 63(5), p. 27 (1985).

The term “substituted” means that a hydrogen group has been replaced with a hydrocarbyl group, a heteroatom, or a heteroatom containing group. For example, 2,5-dimethyl furan is a furan group substituted with a methyl group at the 2 position and at the 5 position.

The terms “hydrocarbyl radical,” “hydrocarbyl,” and “hydrocarbyl group” are used interchangeably throughout this document. For purposes of this disclosure, “hydrocarbyl radical” is defined to be C₁ to C₂₀ radicals, that may be linear, branched, or cyclic (aromatic or non-aromatic), for example methyl, ethyl, ethenyl, and isomers of propyl, butyl, pentyl, hexyl, heptyl, octyl, nonyl, decyl, undecyl, and or dodecyl. An “alpha-olefin” is an olefin having a double bond at the alpha (or 1-) position and examples of α-olefins include ethylene, propylene, 1-butene, 1-pentene, 1-hexene, 1-heptene, 1-octene, 1-nonene, 1-decene, 1-undecene, and 1-dodecene.

5-hydroxymethylfurfural (HMF) is represented by the formula:

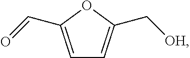

and can be obtained by the dehydration of fructose, as illustrated in Route I. 2,5-bis hydroxymethylfuran (BHMF) is represented by the formula:

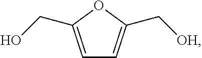

and can be obtained by hydrogenation of HMF, as follows:

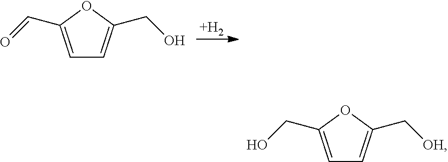

as disclosed in “Catalytic Hydrogenation over Platinum Metals,” P. N. Rylander, Academic Press, New York, 1967, pp. 246-249. 2,5-diformyl furan is represented by the formula:

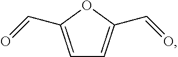

and can be obtained by oxidation of HMF, as follows:

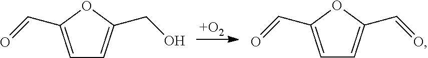

as disclosed in Green Chemistry, 2012, 14, pp. 2986-2989 among others. Dimethyl furan (DMF) is represented by the formula:

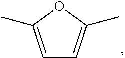

and can be obtained by the dehydration of fructose to form HMF, followed by hydrogenation and dehydration of the HMF, as illustrated in Route II. The various catalysts and conditions for these reactions are well-known in the art and need not be repeated herein.

The following abbreviations may be used through this specification: Me is methyl, MeOH is methanol, HMBA is 4-(hydroxymethyl)benzaldehyde, DHMB is 1,4-dihydroxymethylbenzene, TPal is terephthalaldehyde, DMTP is dimethylterephthalate, HMBA is 4-(hydroxymethylbenzaldehyde, PDM is 1,4-phenylenedimethanol, Ph is phenyl, RT is room temperature which is defined as 25° C. unless otherwise specified, and tol is toluene.

The present invention is associated with processes for the conversion of substituted furan (SF) to bicyclic ether, which is then dehydrated to form a substituted phenyl which is then oxidatively esterified to dimethylterephthalate. Preferably, less than two molecules of hydrogen are added per SF molecule (preferably less than 1.5 molecules, preferably less than 1 molecule, preferably the SF is not hydrogenated) prior to conversion to the bicyclic ether. Alternately less than two moles of hydrogen are added per mole of SF, preferably less than 1.5 moles, preferably less than 1 mole, preferably the SF is not hydrogenated, prior to conversion to the bicyclic ether. More particularly, the cycloaddition of ethylene to an SF, such as HMF, 2,5-diformyl furan, DMF or BHMF, followed by dehydration of the bicyclic ether formed, then oxidative esterification, can be used to produce dimethylterephthalate in good yields as well as save on production costs as costly hydrogenation step(s) are reduced or eliminated.

In a preferred embodiment, this invention relates to a dimethylterephthalate production process comprising reacting substituted furan with ethylene under cycloaddition reaction conditions and in the presence of a catalyst (such as activated carbon, acid washed activated carbon, silica, alumina, a zeolitic molecular sieve, or a non-zeolitic molecular sieve) to produce a bicyclic ether, which is then dehydrated to form a substituted phenyl and thereafter oxidatively esterifying the substituted phenyl to dimethylterephthalate, wherein the substituted furan is represented by the formula:

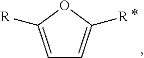

the bicyclic ether is represented by the formula:

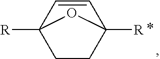

and the substituted phenyl is represented by the formula:

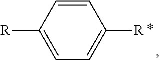

where R and R* are separately selected from CH₃, HC═O and —CH₂OH. In a preferred embodiment of the invention, R and R* are the same. In another embodiment of the invention, R and R* are different. In a preferred embodiment of the invention, R is —CH₂OH and R* is HC═O. In another embodiment of the invention, both R and R* are —CH₂OH or both R and R* are HC═O. In another embodiment of the invention, both R and R* are CH₃.

In a preferred embodiment of the invention, less than two moles of hydrogen are added per SF molecule prior to the ethylene cycloaddition step, preferably less than 1.5 moles, preferably less than 1 mole, preferably the SF is not hydrogenated prior to the cycloaddition step.

As shown below, the cycloaddition of ethylene results in an intermediate bicyclic ether compound, which subsequently dehydrates to the 1,4-disubstituted phenyl ring. The 1,4-disubstituted phenyl can then be oxidatively esterified in MeOH into dimethylterephthalate:

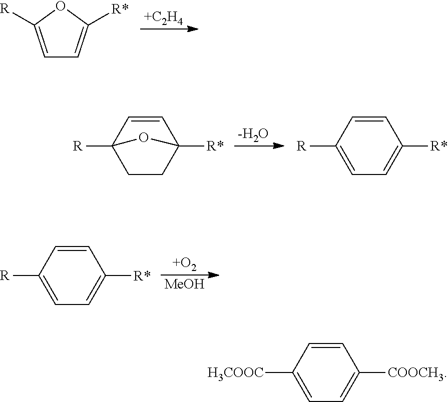

In a preferred embodiment of the invention, the substituted furan is represented by the formula:

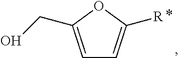

and/or the bicyclic ether is represented by the formula:

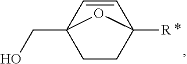

and/or the substituted phenyl is represented by the formula:

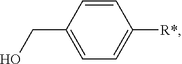

where R* is CH₃, HC═O or —CH₂OH, preferably HC═O.

Thus, according to one embodiment, the reaction pathway is:

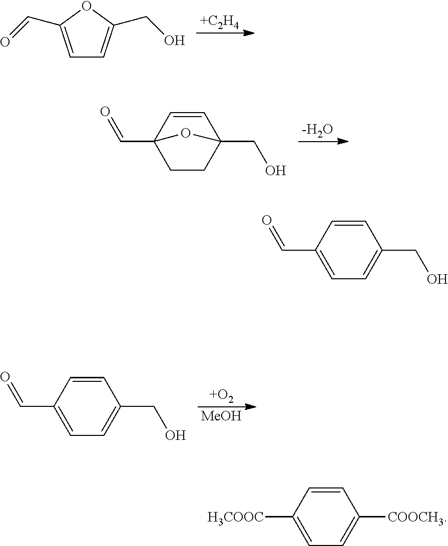

According to another embodiment, the reaction pathway is:

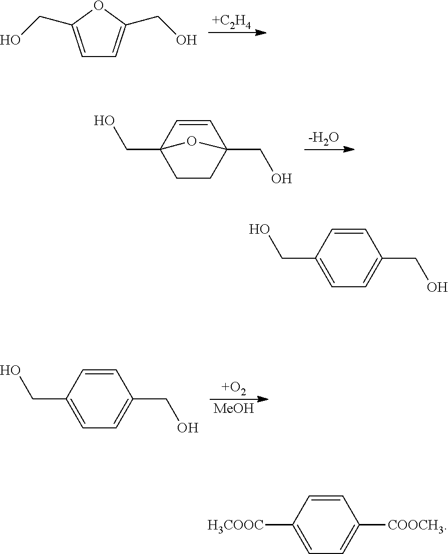

In an alternative embodiment, the reaction pathway is:

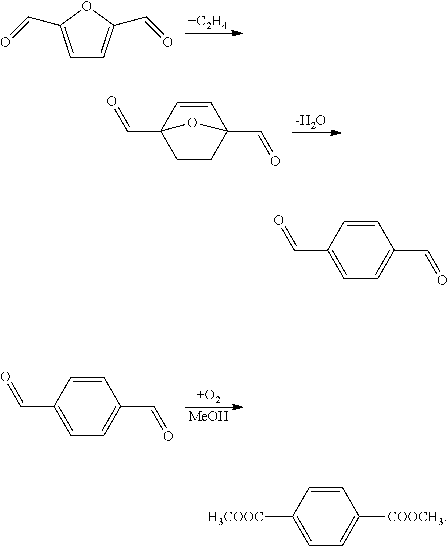

Alternatively, the reaction pathway is:

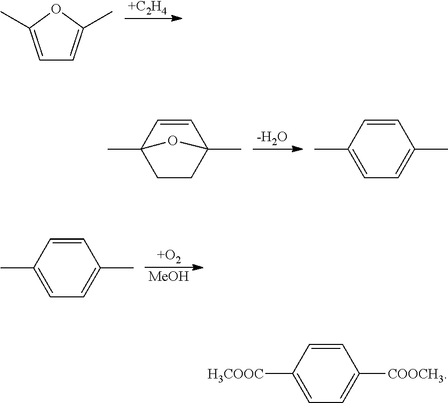

Advantageously, the SF (such as BHMF, 2,5-diformyl furan, DMF or HMF) starting material for the processes may be synthesized from carbohydrates, thereby providing a production route to dimethylterephthalate that relies at least partly on renewable feedstocks. For example, the use of glucose or fructose as a source of SF, such as BHMF, 2,5-diformyl furan, DMF or HMF, results in a process in which 6 of the 8 (75%) dimethylterephthalate carbon atoms originate from a carbohydrate. Moreover, if the ethylene used as a reactant in processes according to the invention is obtained from biomass ethanol, then the dimethylterephthalate produced is completely derived (i.e., all 8 of its 8 carbon atoms) from renewable feedstock.

The use of a solvent is useful for the formation of HMF from sugars, as well as for the ethylene cycloaddition reaction. For example, Leshkov, Y. R. et al. report the production of 5-hydroxymethylfurfural (HMF) in high yields by the acid catalyzed dehydration of fructose utilizing a biphasic reaction scheme with solvent extraction of the HMF product from the aqueous reaction media, for example with butanol as the solvent (NATURE, June 2007, (447): pp. 982-5). In the ethylene cycloaddition reaction, Chang et al. report the use of hexane solvent in the reaction of ethylene with DMF to produce p-xylene (GREEN CHEMISTRY (2013) DOI: 10.1039/c3 gc40740c). In an additional embodiment, the same solvent is used in the HMF production step as in the ethylene cycloaddition step. When the HMF is hydrogenated (with less than two moles of hydrogen per mole of HMF), the same solvent is used in all three steps. In a further embodiment, the solvent used is the same material produced in the ethylene cycloaddition and dehydration reaction. Useful solvents include methanol, butanol, hexane, toluene, methyl isobutyl ketone, HMBA, PDM and the like.

In the cycloaddition/dehydration step, the presence of water in the reaction mixture can be detrimental, as it can hydrolize the furan ring and/or slow or limit the dehydration reaction. In another embodiment, water is continuously removed from the reaction mixture by circulating excess ethylene through the reacting fluid, condensing and separating water from the gaseous ethylene effluent, and returning the unreacted ethylene vapor to the reaction mixture.

Embodiments of the invention are directed to dimethylterephthalate production processes comprising reacting substituted furan, such as BHMF, 2,5-diformyl furan, DMF or HMF, with ethylene under cycloaddition reaction conditions, preferably in the presence of a catalyst to produce a bicyclic ether, which is then dehydrated to produce a substituted phenyl which is then oxidatively esterified to dimethylterephthalate. Representative cycloaddition reaction conditions include a temperature from about 100° C. (212° F.) to about 300° C. (572° F.), an ethylene partial pressure from about 1000 kPa (145 psig) to about 10,000 kPa (14500 psig), and a reactor residence time from about 1 hour to about 48 hours. The processes may be performed batch-wise or in a continuous manner, for example by passing the SF, such as BHMF, 2,5-diformyl furan, DMF or HMF and ethylene reactants continuously over a fixed bed of catalyst. A representative catalyst is activated carbon (e.g., in a solid, powder form), and particularly carbon that has been activated by washing with an acid such as H₃PO₄. Other solid materials, and particularly those having a high surface area (e.g., zeolitic or non-zeolitic molecular sieves) and/or adsorptive capacity for the aromatic and olefinic feed components, may also be used as catalysts. Any of these catalysts may optionally be promoted with an alkali or alkaline earth metal (group 1 or 2) promoter.

The cycloaddition reaction conditions and catalyst can provide at least about 50% conversion of the SF, such as BHMF, 2,5-diformyl furan, DMF or HMF, with dimethylterephthalate representing at least about 60%, on a molar basis, of the converted furan (i.e., at least about 60% selectivity to dimethylterephthalate, or at least about 0.6 moles of dimethylterephthalate produced for each mole of SF converted).

Therefore, according to embodiments of the invention, the conversion of a hexose such as glucose or fructose to SF, such as BHMF, 2,5-diformyl furan, DMF or HMF followed by cycloaddition, then oxidative esterification to dimethylterephthalate provides a basis for dimethylterephthalate production using at least one renewable carbohydrate feedstock. Particularly useful embodiments of the invention are directed to carbohydrate based processes for producing terepthalic acid comprising converting a hexose such as glucose or fructose to BHMF, 2,5-diformyl furan, DMF or HMF and then cycloaddition with ethylene to produce a substituted phenyl, which is then oxidatively esterified to dimethylterephthalate.

Without being bound by theory, the reaction is believed to proceed through the Diels-Alder cycloaddition of ethylene to the furan ring of BHMF, 2,5-diformyl furan, DMF or HMF, followed by ring opening with the elimination of water (dehydration) to generate a bisubstituted phenyl. Suitable catalysts and reaction conditions can improve productivity or yield, especially compared to thermal or non-catalytic reactions. The terms “catalyst” and “catalytic” are meant to encompass agents that reduce the activation energy needed for a desired reaction, as well as promoters that enhance the effectiveness of such agents.

Suitable catalysts include carbon and particularly activated carbon having a high surface area, for example of at least about 700 square meters per gram (m²/gram), as measured according to the BET method (ASTM 6556-09). Generally, the surface area is in the range from about 700 to about 3000 m²/gram and often from about 700 to about 1500 m²/gram. Catalysts of particular interest include carbon that is activated by washing with an acid, for example, phosphoric acid, to provide the high surface area in these representative ranges and a possibly a number of other desirable properties. Such properties include a total oxygen content of at least about 1% by weight (e.g., in the range from about 1% to about 20%, and often from about 1% to about 10%, by weight).

Thermal processing or activation can also be used to obtain porous carbon particles having a large internal surface area. Regardless of whether the activation is performed chemically or thermally, the activated carbon particles may be granular, spherical, pelletized, or powdered, as supplied by a number of commercial manufacturers, including Norit Americas, Inc. (Marshall, Tex. USA), Japan EnviroChemicals (Tokyo, Japan), Jacobi Carbons AB (Kalmar, Sweden), and Calgon Carbon Corporation (Pittsburgh, Pa.). A representative average particle size of a powdered activated carbon that is used in the methods described herein is less than about 300 microns (50 mesh) and often in the range from about 50 microns (300 mesh) to about 300 microns (50 mesh). Screening may be used in some cases to achieve a desired average particle size.

In general, the activated carbon is derived from an organic source, such as wood, ground coconut shells, etc. Various forms of activated carbon include a surface oxidized activated carbon, a graphite, a graphite oxide, or a carbon nanomaterial. Carbon nanomaterials include, but are not limited to, carbon nanotubes, carbon nanohorns, carbon nanofibers, Fullerenes, etc. Activated carbon materials also include those having one or more surface modifications, for example, performed by covalently bonding of acidic or basic materials to control acidity and/or by the incorporation of one or more metals that are catalytically active for the conversion of adsorbed organic compounds. Such surface modifications can therefore supplement (promote) the catalytic activity of the activated carbon for the desired conversion.

In addition to activated carbon, a number of other materials having a relatively high BET surface area (e.g., at least about 200 m²/gram, and often in the range from about 200 m²/gram to about 500 m²/gram), as well as having sufficient capacity for the adsorption of organic reactants, may be used as solid catalysts. These materials include inorganic oxides such as silica (e.g., in the form of a silica gel), alumina, zirconia, etc., as well as zeolitic molecular sieves and non-zeolitic molecular sieves. Zeolitic molecular sieves suitable for use as catalysts are crystalline aluminosilicates which in the calcined form may be represented by the general formula:

M₂/ₙO:Al₂O₃:xSiO₂:yH₂O

where M is a cation, such as H, alkaline metals (group 1, preferably Na, K, etc.), alkaline earth metals (group 2, preferably Mg, Ca, etc.) rare earth metals (group 3, preferably La, Y, etc.), and transition metals (groups 4-12), and NH₄, n is the valence of the cation, x has a value of from about 5 to 100, and y has a value of from about 2 to 10. Zeolites are described in detail by D. W. Breck, Zeolite Molecular Sieves, John Wiley and Sons, New York (1974), and elsewhere. In useful embodiments, the catalyst comprises a large pore zeolite, such as Y, zeolite β, mordenite, ZSM-12, ZSM-18, MCM-22, and/or MCM-49, and/or medium pore zeolites, such as ZSM-5, ZSM-11, ZSM-23, ZSM-48, and ZSM-57.

In useful embodiments, the catalyst comprises a zeolite, such as ZSM-5, zeolite beta, ITQ-13, MCM-22, MCM-49, ZSM-11, ZSM-12, ZSM-22, ZSM-23, ZSM-35, and ZSM-48, ZSM-57, preferably having been modified by steaming so as to have a Diffusion Parameter for 2,2 dimethylbutane of about 0.1 to 15 Sec⁻¹ when measured at a temperature of 120° C. and a 2,2 dimethylbutane pressure of 60 torr (8 kPa). Alternately, the catalyst may comprise ZSM-5, MCM-22, PSH-3, SSZ-25, ERB-I, ITQ-I, ITQ-2, ITQ-13, ITQ-39, MCM-36, MCM-49, MCM-56, Zeolite X, Zeolite Y, Zeolite Beta, and the like. Diffusion Parameter is defined at paragraph [0033] of WO 2013/009399.

Non-zeolitic molecular sieves include molecular sieves that are of the chemical composition, on an anhydrous basis, expressed by the empirical formula:

(ELxAl_{y}Pz)q₂

where EL is an element selected from the group consisting of silicon, magnesium, zinc, iron, cobalt, nickel, manganese, chromium and mixtures thereof, x is the mole fraction of EL and is at least 0.005, y is the mole fraction of Al and is at least 0.01, z is the mole fraction of P and is at least 0.01, x+y+z=1, and q is oxygen. When EL is a mixture of metals, x represents the total amount of the element mixture present. Preferred elements (EL) are silicon, magnesium and cobalt, with silicon being especially preferred. These non-zeolitic molecular sieves are also referred to as “ELAPOs”. The preparation of various ELAPOs are known in the art and described, for example, in U.S. Pat. No. 7,317,133, U.S. Pat. No. 5,191,141, U.S. Pat. No. 4,554,143, U.S. Pat. No. 4,440,871, U.S. Pat. No. 4,853,197, U.S. Pat. No. 4,793,984, U.S. Pat. No. 4,752,651, and U.S. Pat. No. 4,310,440.

As indicated above, any of the above solid catalysts may incorporate a metal promoter having catalytic activity for the desired conversion. Representative metals include alkali and alkaline earth metals (groups 1 and 2), as well as rare earth (group 3, preferably lanthanides plus scandium and yttrium) and transition metals (groups 3-12, preferably 4-12). Combinations of two or more metals may be used in conjunction with any of the solid catalysts described above (e.g., as support materials).

Alternately, any catalyst disclosed in ACS Catalysis, 2012, 2, pp. 935-939 may be used herein.

The reaction of BHMF, 2,5-diformyl furan, DMF or HMF with ethylene proceeds in the presence of a catalyst as discussed above under suitable cycloaddition reaction conditions. Advantageously, the use of solvents (e.g., dimethylsulfoxide) that do not participate the desired reaction pathway can be minimized or even eliminated. According to some embodiments, therefore, the cycloaddition reaction conditions include a reaction mixture that is solvent-free or substantially solvent-free (i.e., contains less than about 10%, less than about 5%, or even less than about 1% of a solvent). Exemplary temperatures in the reactor or reaction zone in which the catalyst is disposed (e.g., in a batch reactor or as a fixed or moving bed in a continuous reaction system) are in the range from about 100° C. (212° F.) to about 300° C. (572° F.), and often from about 150° C. (302° F.) to about 225° C. (437° F.). Favorable cycloalkylation reaction conditions also include an ethylene partial pressure of at least about 1000 kPa (145 psig), generally in the range from about 1000 kPa (145 psig) to about 10,000 kPa (1450 psig), and often in the range from about 2000 kPa to about 5000 kPa. The total pressure is typically from about 2% to about 50% higher than the ethylene partial pressure, due to the contributions, to the overall pressure in the reactor or reaction zone, of (i) the vapor pressure of the SF (i.e. BHMF, 2,5-diformyl furan, DMF or HMF) at the reaction temperature, and/or (ii) possible diluents and/or impurities (e.g., ethane).

Whether the reaction is carried out batchwise or continuously, the cycloaddition reaction conditions also generally include a reactor residence time in the range from about 1 hour to about 48 hours, and normally from about 3 hours to about 30 hours. The reactor residence time, however, may be significantly reduced in the case of a continuous process in which unconverted SF and/or ethylene are recycled to provide a relatively high overall conversion, even if the per-pass conversion is significantly less. Reactant SF may be continuously fed to a cycloaddition reaction zone, for example, at a liquid hourly space velocity (LHSV) from about 0.05 hr⁻¹ to about 5 hr⁻¹. As is understood in the art, the Liquid Hourly Space Velocity (LHSV, expressed in units of hr⁻¹) is the volumetric liquid flow rate over the catalyst bed divided by the bed volume and represents the equivalent number of catalyst bed volumes of liquid processed per hour. The LHSV is therefore closely related to the inverse of the reactor residence time.

The Diels-Alder cycloaddition of ethylene to the 2,5-disubstituted furan is facilitated if the substituents are both electron-donating groups. In 5-hydroxymethyl furfural, one of the substituents is a carbaldehyde, which is electron-withdrawing, while the other is an electron-donating hydroxymethyl group. Selective addition of one molecule of hydrogen to the carbaldehyde group on the HMF molecule will convert the electron-withdrawing carbonyl to an electron-donating hydroxymethyl group. The resulting 2,5-bis hydroxymethyl furan (BHMF) is more reactive than the 5-hydroxymethyl furfural (HMF) in the Diels-Alder cycloaddition reaction with ethylene as the dienophile, and the BHMF or HMF as the diene. The selective hydrogenation of furan-2-carbaldehyde (or furfural) to 2-furanmethanol (or furfuryl alcohol) is known to those in the art of the production of furfuryl alcohol. Early work by Kaufmann and Adams report high conversion and selectivity of furfural to furfuryl alcohol over reduced platinum catalysts (J. Am. Chem. Soc., December 1923, pp. 3029-3044). Conversion over nickel and palladium catalysts were also reported. In more recent work by Sharma et al, 100% conversion of furfural to furfuryl alcohol with 96% selectivity is reported over Cu:Zn:Cr:Zr catalyst (App. Cat A: Gen 454, pp. 127-136 (2013) DOI: 10.1016/j.apcata.2012.12.010). Similar catalysts and conditions may be used to convert 5-hydroxymethyl furfural to 2,5-bis hydroxymethyl furan.

In an exemplary continuous process, the reactants SF and ethylene are continuously fed to one or more reactors containing a fixed bed of the catalyst (e.g., in a swing-bed reactor system having multiple fixed bed reactors), and a product comprising the converted 1,4-disubstituted phenyl (such as HMBA, TPal, p-xylene or PDM) is continuously withdrawn together with unconverted reactants and reaction byproducts. The unconverted materials are preferably separated, for example, based on differences in their relative volatility using one or more separation operations (e.g., flash separation or distillation) employing a single stage or multiple stages of vapor-liquid equilibrium contacting.

According to a specific embodiment, unconverted ethylene, together with low boiling byproducts and impurities, is separated from the cycloaddition reaction zone effluent using a single-stage flash separation. The liquid bottoms product of this flash separation is then passed to at least one multi-stage distillation column to separately recover purified 1,4 disubstituted phenyl and unconverted SF. The unconverted SF and/or unconverted ethylene may be recycled to the cycloaddition reaction zone, optionally after purging a portion of either or both of these streams to limit the accumulation of byproducts having similar boiling points. In a particular embodiment, excess ethylene is added to the reactor to strip water from the reaction zone; this water is condensed and separated from the ethylene before being recycled to the reactor. According to a particular continuous operation, the flow rate of ethylene reactant to the cycloaddition reactor or reaction zone is controlled to maintain a desired total pressure. Such an operation based on pressure demand ensures that ethylene is fed at a rate that matches essentially its consumption plus losses due to dissolution and possibly a gas purge (vent).

Whether a batch or a continuous process is used for the catalytic conversion of SF to substituted phenyl, the cycloaddition reaction conditions generally provide a SF conversion (which may be a per-pass conversion in the cycloaddition reaction zone, in the case of operation with the recycle of unconverted SF) of at least about 50%, for example from about 50% to about 90% and often from about 50% to about 75%. The recycle of unconverted SF, for example to extinction or nearly extinction, can provide an overall conversion that is complete or nearly complete. Of the converted SF, the selectivity to substituted phenyl is generally at least about 60%, meaning that at least about 0.6 moles of substituted phenyl are produced for each mole of SF converted. Typical selectivities to substituted phenyl are from about 60% to about 95%. In view of these representative conversion and selectivity values, the overall yield of substituted phenyl is generally at least about 30%, typically from about 30% to about 90%, and often from about 90% to about 75%, of the theoretical yield based on complete conversion of SF with a stoichiometric amount (1:1 molar) of ethylene to substituted phenyl and no byproduct formation.

After the SF is combined with the ethylene and the addition catalyst, a bicyclic ether is formed. That ether is then preferably dehydrated to form the substituted phenyl. The bicyclic compound can go through dehydration in the same reaction step and in the presence of the same catalyst, and at the same conditions as the ethylene cycloaddition reaction.

After the SF is combined with the ethylene and the addition catalyst, a bicyclic ether is formed. That ether is then preferably dehydrated to form the substituted phenyl which is then oxidatively esterified to form dimethylterephthalate. The substituted phenyl can be oxidatively esterified to dimethylterephthalate using a process wherein the substituted phenyl is dissolved in MeOH and an oxidant, such as oxygen or air is passed into contact with the solution in the presence of an oxidation esterification catalyst.

For example, one embodiment of a 1,4-disubstituted phenyl is dissolved in MeOH, and the solution is placed into a reactor with an oxidative esterification catalyst. The reactor is pressurized with an oxidant (such as oxygen or air) up to about 1 bar (100 kPa), or even up to about 2 bar (200 kPa), or even up to about 10 bar (1000 kPa), and subjected to a temperature between from about −20° C. to about 250° C., or from about −20° C. to about 100° C., depending on the pressure inside the reactor. Advantageously, the reaction may be conducted at relatively lower temperatures, such as from about −20° C. to about 80° C., or from about 0° C. to about 70° C., or even from about 10° C. to about 60° C., or even from about 20° C. to about 50° C. The reaction may be conducted at a substantially neutral pH, or in the presence of a recoverable solid base catalyst.

The oxidative esterification reaction is more facile with carbonyl or hydroxymethyl substitutions on the phenyl ring, relative to the methyl substituents in p-xylene, and the more facile reactivity of carbonyl or hydroxymethyl substituents will be translated to higher selectivity to dimethylterephthalate.

The oxidative esterification catalyst may be a heterogeneous catalyst bearing metal including but not limited to cobalt, gold, palladium, platinum, and combinations thereof. The oxidative esterification catalyst may be supported on materials including but not limited to carbon, alumina, titania, silica, and metal-organic framework, and may have ligands, such as nitrogen ligands. For example, Jagadeesh et al. (“Selective Oxidation of Alcohols to Esters Using Heterogeneous Co₃O₄—N@C Catalysts under Mild Conditions”, Journal of the American Chemistry Society, 2013, 135, pp. 10776-10782) discloses oxidation of DHMB to DMTP in 89% yield using Co catalyst supported on carbon material, methanol as solvent/reactant, in combination with 0.4 eq. of K₂CO₃ base. The reaction was conducted under 1 bar O₂ at 60° C. for 24 hr.

In an alternative embodiment, the MeOH solvent used in the oxidative esterification could be replaced with other longer-chain alcohols, which would result in a terephthalate ester having longer-chain ester moieties. Even more preferably, oxo-alcohols derived from hydroformylation of olefins could be used as solvents. An “OXO-alcohol” is an organic alcohol, or mixture of organic alcohols, which is prepared by hydroformylating an olefin, followed by hydrogenation to form the alcohols. Typically, the olefin is formed by light olefin oligomerization over heterogenous acid catalysts, which olefins are readily available from refinery processing operations. The reaction results in mixtures of longer-chain, branched olefins, which subsequently form longer chain, branched alcohols, as described in U.S. Pat. No. 6,274,756, incorporated herein by reference in its entirety. The OXO-alcohols consist of multiple isomers of a given chain length due to the various isomeric olefins obtained in the oligomerization process, in tandem with the multiple isomeric possibilities of the hydroformylation step.

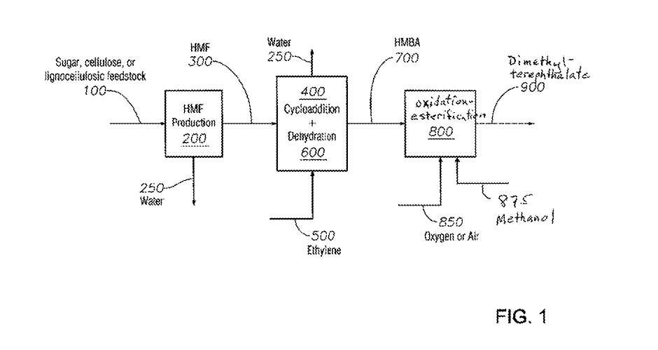

FIG. 1 shows a preferred embodiment of the invention. A feedstock (100), preferably a renewable feedstock, such as sugar, cellulose, or lignocellulose is first converted to a 2,5-disubstituted furan compound (200) such as 5-hydroxymethyl furfural (HMF) (300), giving off water (250). The HMF is then subjected to cycloaddition conditions (400) with ethylene (500) to form a bicyclic ether intermediate, which subsequently dehydrates (600), giving off water (250) to a 1,4-disubstituted phenyl such as 4-(hydroxymethyl) benzaldehyde (HMBA)(700). This product is subjected to oxidative esterification (800) in the presence of oxygen or air (850) and methanol (875) to produce dimethylterephthalate (900).

The dimethylterephthalate is useful for preparing polyesters such as polyethylene terephthalate polymer (PET) using processes well known in the art. Once manufactured, the PET can be processed so as to produce a thermoplastic PET resin used in synthetic fibers, beverage, food and other liquid containers; thermoforming applications; and engineering resins often in combination with glass fiber.

In another embodiment of the invention, when R and R* on the substituted phenyl contain OH groups, the substituted phenyl can be hydrogenated to the cycloalkane and optionally used as a monomer in the production of polyester.

In particular, the 1,4-cyclohexanedimethanol is one of the most important co-monomers for production of polyethyleneterephthalate (PET). Currently, 1,4-cyclohexanedimethanol is produced via hydrogenation of terephthalic acid esters such as dimethylterephthalate at high temperature and high pressure.

In another embodiment this invention relates to:
1. A dimethylterephthalate production process comprising:

reacting substituted furan with ethylene under cycloaddition reaction conditions and in the presence of a cycloaddition catalyst to produce a bicyclic ether;

wherein the substituted furan is represented by the formula:

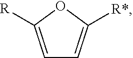

wherein R and R* are separately selected from CH₃, HC═O and —CH₂OH, and the bicyclic ether is represented by the formula:

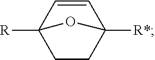

dehydrating the bicyclic ether to produce a substituted phenyl of the formula:

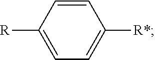

dissolving said substituted phenyl in methanol; and

oxidizing and esterifying the substituted phenyl in the presence of an oxidative esterification catalyst to form dimethylterephthalate.
2. The process of paragraph 1, wherein both R and R* are HC═O, or both R and R* are —CH₂OH, or both R and R* are CH₃.
3. The process of paragraph 1, wherein one of R and R* is HC═O and the other is —CH₂OH.
4. The process of paragraph 1, wherein one of R and R* is CH₃ and the other is —CH₂OH.
5. The process of paragraph 1, wherein one of R and R* is CH₃ and the other is HC═O.
6. The process of any of the preceding paragraphs, wherein said oxidative esterification is conducted at a temperature from −50° C. to 100° C.
7. The process of any of the preceding paragraphs, wherein the oxidative esterification catalyst is a heterogeneous catalyst bearing metal including but not limited to cobalt, gold, palladium, platinum, and combinations thereof.
8. The process of any of the preceding paragraphs, wherein the oxidative esterification catalyst is supported on materials including but not limited to carbon, alumina, titania, silica, and metal-organic framework.
9. The process of any of the preceding paragraphs, wherein said oxidative esterification is conducted at a substantially neutral pH or in the presence of a recoverable solid base catalyst.
10. The process of any of the preceding paragraphs, wherein said substituted furan is derived from conversion of glucose or fructose.
11. The process of any of the preceding paragraphs, which does not include oxidizing the substituted phenyl to form terephthalic acid.
12. A carbohydrate based process for producing dimethylterephthalate comprising:
   (a) converting a hexose to 5-hydroxymethylfurfural;
   (b) reacting the 5-hydroxymethylfurfural with ethylene under cycloaddition reaction conditions and in the presence of a catalyst to produce a bicyclic ether, then dehydrating the bicyclic ether to produce a compound represented by the formula (I):

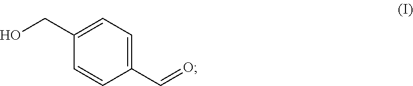

and
(c) dissolving the compound represented by the formula (I) in methanol and contacting the solution with oxygen in the presence of an oxidative esterification catalyst to produce dimethylterephthalate,
wherein the compound of formula (I) is not converted to terephthalic acid prior to step (c).
13. A carbohydrate based process for producing dimethylterephthalate comprising:
   (a) converting a hexose to 5-hydroxymethylfurfural;
   (b) hydrogenating 5-hydroxymethylfurfural to form 2,5-bis hydroxymethylfuran;
   (c) reacting the 2,5-bis hydroxymethylfuran with ethylene under cycloaddition reaction conditions and in the presence of a catalyst to produce a bicyclic ether, then dehydrating the bicyclic ether to produce a compound represented by the following formula (II):

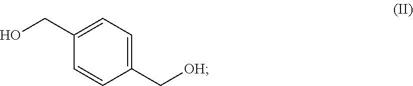

and
(d) dissolving the compound represented by the formula (II) in methanol and contacting the solution with oxygen in the presence of an oxidation/esterification catalyst to produce dimethylterephthalate,
wherein the compound of formula (II) is not converted to terephthalic acid prior to step (d).
14. A carbohydrate based process for producing dimethylterephthalate comprising:
   (a) converting a hexose to 5-hydroxymethylfurfural;
   (b) partially oxidizing the 5-hydroxymethylfurfural to form diformyl furan;
   (c) reacting the diformyl furan with ethylene under cycloaddition reaction conditions and in the presence of a catalyst to produce a bicyclic ether, then dehydrating the bicyclic ether to produce a compound represented by the following formula (III):

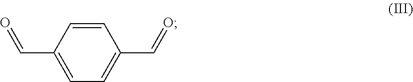

and
(d) dissolving the compound represented by the formula (III) in methanol and contacting the solution with oxygen in the presence of an oxidative esterification catalyst to produce dimethylterephthalate,
wherein the compound of formula (III) is not converted to terephthalic acid prior to step (d).
15. A carbohydrate based process for producing dimethylterephthalate comprising:
   (a) converting a hexose to 5-hydroxymethylfurfural;
   (b) reducing the 5-hydroxymethylfurfural to form dimethyl furan;
   (c) reacting the dimethyl furan with ethylene under cycloaddition reaction conditions and in the presence of a catalyst to produce a bicyclic ether, then dehydrating the bicyclic ether to produce a compound represented by the following formula (IV):

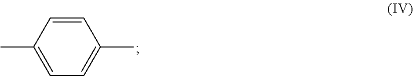

and
(d) dissolving the compound represented by the formula (IV) in methanol and contacting the solution with oxygen in the presence of an oxidative esterification catalyst to produce dimethylterephthalate,
wherein the compound of formula (IV) is not converted to terephthalic acid prior to step (d).

### EXPERIMENTAL

Room temperature is about 23° C., unless otherwise noted.

### PROPHETIC EXAMPLE 1

### Non-Catalytic Conversion of HMF to HMBA

100 mL of 1.0M HMF in 2-butanol is charged to an autoclave having a volume of 160 mL fitted with a gas inlet, thermocouple, pressure transducer, and magnetic stir bar. The autoclave is sealed, pressurized at room temperature with ethylene, and heated to a reaction temperature of 250° C. The reaction is allowed to run for a 24-hour reaction period while maintaining ethylene pressure in the autoclave of 6200 kPa. The reactor is then cooled and an analysis of the products is obtained.

### PROPHETIC EXAMPLE 2

### Non-Catalytic Conversion of BHMF to PMB

100 mL of 1.0M BHMF in 2-butanol is charged to an autoclave having a volume of 160 mL fitted with a gas inlet, thermocouple, pressure transducer, and magnetic stir bar. The autoclave is sealed, pressurized at room temperature with ethylene, and heated to a reaction temperature of 250° C. The reaction is allowed to run for a 24-hour reaction period while maintaining ethylene pressure in the autoclave of 6200 kPa. The reactor is then cooled and an analysis of the products is obtained.

### PROPHETIC EXAMPLE 3

### Catalytic Conversion of HMF to HMBA

The experimental procedure described in Prophetic Example 1 is followed, except that 0.5 g of solid catalyst in a particle form (granular or powdered) is added to the reactor prior to pressurization with ethylene. The experiments are described in Table 1.

TABLE 1

| Example | Catalyst | Si/Al ratio | Reaction temp (° C.) | Reactant |
|---|---|---|---|---|
| A | H-BEA | 12.5 | 250 | HMF |
| B | H-BEA | 19 | 250 | HMF |
| C | H-FAU | 2.6 | 250 | HMF |
| D | H-ZSM-5 | 15 | 250 | HMF |
| E | Niobic acid | | 250 | HMF |
| F | γ-Al₂O₃ | | 250 | HMF |

### PROPHETIC EXAMPLE 4

### Catalytic Conversion of BHMF to PDM

The experimental procedure described in Prophetic Example 2 is followed, except that 0.5 g of solid catalyst in a particle form (granular or powdered) is added to the reactor prior to pressurization with ethylene. The experiments are described in Table 2.

TABLE 2

| Example | Catalyst | Si/Al ratio | Reaction temp (° C.) | Reactant |
|---|---|---|---|---|
| A | H-BEA | 12.5 | 250 | BHMF |
| B | H-BEA | 19 | 250 | BHMF |
| C | H-FAU | 2.6 | 250 | BHMF |
| D | H-ZSM-5 | 15 | 250 | BHMF |
| E | Niobic acid | | 250 | BHMF |
| F | γ-Al₂O₃ | | 250 | BHMF |

### PROPHETIC EXAMPLE 5

### Catalytic Hydrogenation of HMF to BHMF

100 mL of 1.0M BHMF in 2-butanol is charged to an autoclave having a volume of 160 mL fitted with a gas inlet, thermocouple, pressure transducer, and magnetic stir bar. 0.5 g of 1% Pt on γ-Al₂O₃ catalyst is added to the autoclave. The autoclave is sealed, pressurized at room temperature with hydrogen, and heated to a reaction temperature of 150° C. The reaction is allowed to run for a while maintaining hydrogen pressure in the autoclave of 2000 kPa. The reactor is stopped by cooling and depressuring when one equivalent of hydrogen is consumed. An analysis of the products is then obtained.

### PROPHETIC EXAMPLE 6

### Catalytic Conversion of BHMF to PDM in Various Solvents

100 mL of 1.0M BHMF in solvent (see Table 3) is charged to an autoclave having a volume of 160 mL fitted with a gas inlet, thermocouple, pressure transducer, and magnetic stir bar. 0.5 g of H-BEA catalyst having a Si/Al ratio of 12.5 is charged to the reactor. The autoclave is sealed, pressurized at room temperature with ethylene, and heated to a reaction temperature of 250° C. The reaction is allowed to run for a 24-hour reaction period while maintaining ethylene pressure in the autoclave of 6200 kPa. The reactor is then cooled and an analysis of the products is obtained.

TABLE 3

| | | | Reaction | |
|---|---|---|---|---|
| Example | Solvent | Catalyst | temp (° C.) | Reactant |
| A | 2-butanol | H-BEA (12.5) | 250 | BHMF |
| B | toluene | H-BEA (12.5) | 250 | BHMF |
| C | 1:1 toluene:2- | H-BEA (12.5) | 250 | BHMF |
| | butanol | | | |
| D | Methyl isobutyl | H-BEA (12.5) | 250 | BHMF |
| | ketone | | | |
| E | HMBA | H-BEA (12.5) | 250 | BHMF |
| F | PDM | H-BEA (12.5) | 250 | BHMF |

### PROPHETIC EXAMPLE 7

### Catalytic Conversion of HMBA to DMPT

The product of Example 3 is dissolved in MeOH and is charged to a reactor in combination with an oxidative esterification catalyst, such as Co₃O₄—N@C (see Journal of the American Chemistry Society, 2013, 135, pp. 10776-10782), under 1 bar (100 kPa) of air at a temperature of 60° C. for a period of 24 h to form DMPT.

### PROPHETIC EXAMPLE 8

### Catalytic Conversion of PDM to DMPT

The product of Example 4 is dissolved in MeOH and is charged to a reactor in combination with an oxidative esterification catalyst, such as Co₃O₄—N@C (see JACS, 2013, 135, pp. 10776-10782), under 1 bar (100 kPa) of air at a temperature of 60° C. for a period of 24 h to form DMPT.

### PROPHETIC EXAMPLE 9

### Catalytic Conversion of TPal to DMPT

The product of TPal is dissolved in MeOH and is charged to a reactor in combination with an oxidative esterification catalyst, such as Co₃O₄—N@C (see JACS, 2013, 135, pp. 10776-10782), under 1 bar (100 kPa) of air at a temperature of 60° C. for a period of 24 h to form DMPT.

### PROPHETIC EXAMPLE 10

### Catalytic Conversion of p-XYLENE to DMPT

The product of p-xylene is dissolved in MeOH and is charged to a reactor in combination with an oxidative esterification catalyst, such as Co₃O₄—N@C (see JACS, 2013, 135, pp. 10776-10782), under 1 bar (100 kPa) of air at a temperature of 60° C. for a period of 24 h to form DMPT.

All documents described herein are incorporated by reference herein, including any priority documents and/or testing procedures to the extent they are not inconsistent with this text. As is apparent from the foregoing general description and the specific embodiments, while forms of the invention have been illustrated and described, various modifications can be made without departing from the spirit and scope of the invention. Accordingly, it is not intended that the invention be limited thereby. Likewise, the term “comprising” is considered synonymous with the term “including” for purposes of Australian law. Likewise whenever a composition, an element or a group of elements is preceded with the transitional phrase “comprising”, it is understood that we also contemplate the same composition or group of elements with transitional phrases “consisting essentially of,” “consisting of”, “selected from the group of consisting of,” or “is” preceding the recitation of the composition, element, or elements and vice versa. Thus, the term “comprising” encompasses the terms “consisting essentially of,” “is,” and “consisting of” and anyplace “comprising” is used “consisting essentially of,” “is,” or consisting of may be substituted therefor.

What is claimed is:

1. A dimethylterephthalate production process comprising:
reacting substituted furan with ethylene under cycloaddition reaction conditions and in the presence of a cycloaddition catalyst to produce a bicyclic ether;
wherein the substituted furan is represented by the formula: 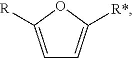
wherein R and R* are separately selected from CH₃, HC═O and —CH₂OH, and the bicyclic ether is represented by the formula: 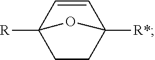
dehydrating the bicyclic ether to produce a substituted phenyl of the formula: 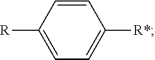
dissolving said substituted phenyl in methanol; and
oxidizing and esterifying the substituted phenyl in the presence of an oxidative esterification catalyst to form dimethylterephthalate.

2. The process of claim 1, wherein both R and R* are HC═O.

3. The process of claim 1, wherein both R and R* are —CH₂OH.

4. The process of claim 1, wherein one of R and R* is HC═O and the other is —CH₂OH.

5. The process of claim 1, wherein both R and R* are CH₃.

6. The process of claim 1, wherein one of R and R* is CH₃ and the other is —CH₂OH.

7. The process of claim 1, wherein one of R and R* is CH₃ and the other is HC═O.

8. The process of claim 1, wherein said oxidative esterification is conducted at a temperature from −50° C. to 100° C.

9. The process of claim 1, wherein the oxidative esterification catalyst is a heterogeneous catalyst bearing metal compromising at least one of cobalt, gold, palladium, platinum, and combinations thereof.

10. The process of claim 9, wherein the oxidative esterification catalyst is supported on materials compromising at least one of carbon, alumina, titania, silica, and metal-organic framework.

11. The process of claim 1, wherein said oxidative esterification is conducted at a substantially neutral pH or in the presence of a recoverable solid base catalyst.

12. The process of claim 1, wherein said substituted furan is derived from conversion of glucose or fructose.

13. The process of claim 1, which does not include oxidizing the substituted phenyl such that it forms terephthalic acid.

14. A carbohydrate based process for producing dimethylterephthalate comprising:
(a) converting a hexose to 5-hydroxymethylfurfural;
(b) reacting the 5-hydroxymethylfurfural with ethylene under cycloaddition reaction conditions and in the presence of a catalyst to produce a bicyclic ether, then dehydrating the bicyclic ether to produce a compound represented by the formula (I): 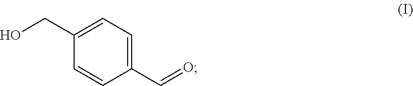
and
(c) dissolving the compound represented by the formula (I) in methanol and contacting the solution with oxygen in the presence of an oxidative esterification catalyst to produce dimethylterephthalate,
wherein the compound of formula (I) is not converted to terephthalic acid prior to step (c).

15. A carbohydrate based process for producing dimethylterephthalate comprising:
(a) converting a hexose to 5-hydroxymethylfurfural;
(b) hydrogenating 5-hydroxymethylfurfural to form 2,5-bis hydroxymethylfuran;
(c) reacting the 2,5-bis hydroxymethylfuran with ethylene under cycloaddition reaction conditions and in the presence of a catalyst to produce a bicyclic ether, then dehydrating the bicyclic ether to produce a compound represented by the following formula (II): 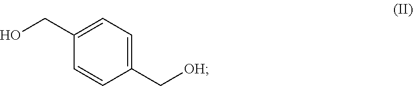
and
(d) dissolving the compound represented by the formula (II) in methanol and contacting the solution with oxygen in the presence of an oxidation/esterification catalyst to produce dimethylterephthalate,
wherein the compound of formula (II) is not converted to terephthalic acid prior to step (d).

16. A carbohydrate based process for producing dimethylterephthalate comprising:
(a) converting a hexose to 5-hydroxymethylfurfural;
(b) partially oxidizing the 5-hydroxymethylfurfural to form diformyl furan;
(c) reacting the diformyl furan with ethylene under cycloaddition reaction conditions and in the presence of a catalyst to produce a bicyclic ether, then dehydrating the bicyclic ether to produce a compound represented by the following formula (III): 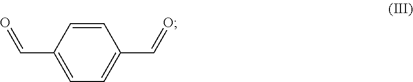
and
(d) dissolving the compound represented by the formula (III) in methanol and contacting the solution with oxygen in the presence of an oxidative esterification catalyst to produce dimethylterephthalate,
wherein the compound of formula (III) is not converted to terephthalic acid prior to step (d).

17. A carbohydrate based process for producing dimethylterephthalate comprising:
(a) converting a hexose to 5-hydroxymethylfurfural;
(b) reducing the 5-hydroxymethylfurfural to form dimethyl furan;
(c) reacting the dimethyl furan with ethylene under cycloaddition reaction conditions and in the presence of a catalyst to produce a bicyclic ether, then dehydrating the bicyclic ether to produce a compound represented by the following formula (IV): 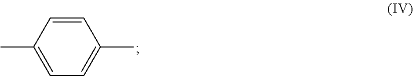
and
(d) dissolving the compound represented by the formula (IV) in methanol and contacting the solution with oxygen in the presence of an oxidative esterification catalyst to produce dimethylterephthalate,
wherein the compound of formula (IV) is not converted to terephthalic acid prior to step (d).
